(19) United States
(12) Patent Application Publication
(10) Pub. No.: US 2011/0015549 A1
(43) Pub. Date: Jan. 20, 2011

(54) METHOD AND APPARATUS FOR TREATING A DISEASED NAIL

(76) Inventors: Shimon Eckhouse, Haifa (IL); Tuvia Dror Kutscher, Shoham (IL); James C. Hsia, Weston, MA (US); Constantinos Demetriou, Nicosia (CY)

Correspondence Address: SMITH FROHWEIN TEMPEL GREENLEE BLAHA, LLC, Two Ravinia Drive, Suite 700, ATLANTA, GA, 30346, US

(21) Appl. No.: 12/892,151

(22) Filed: Sep. 28, 2010

Related U.S. Application Data
(63) Continuation-in-part of application No. 11/229,024, filed on Sep. 16, 2005.
(60) Provisional application No. 60/644,245, filed on Jan. 13, 2005.
(60) Provisional application No. 60/656,356, filed on Feb. 25, 2005.
(60) Provisional application No. 61/318,291, filed on Mar. 27, 2010.
(60) Provisional application No. 61/248,997, filed on Oct. 6, 2009.
(60) Provisional application No. 61/307,517, filed on Feb. 24, 2010.

(51) Int. Cl. A61N 1/04; A61H 1/00; A61N 5/06; A61N 7/02

(52) U.S. Cl. 601/3; 601/2; 607/3; 607/88

(57) ABSTRACT

A diseased nail is treated using electromagnetic radiation and/or other forms of energy that are applied to the diseased area to eliminate, substantially eliminate or effectively reduce the source of disease in the nail. A sensor can be used to ensure proper placement of an applicator that applies the energy to the diseased area of the nail. A temperature monitor can be employed to monitor changes or temperature levels and then adjust the energy application accordingly. In additional, manual forms of adjustment can be used to control the application of energy.

### CROSS-REFERENCE TO RELATED APPLICATIONS

This is a United States non-provisional application being filed under 35 USC 111 and 37 CFR 1.53(b) and is a continuation-in-part of U.S. patent application Ser. No. 11/229,024 filed on Sep. 16, 2005, which application claims the benefit of the priority of U.S. Provisional Application for patent assigned Ser. No. 60/644,245 on Jan. 13, 2005, as well as U.S. Provisional Application for patent assigned Ser. No. 60/656,356, filed on Feb. 25, 2005, each of these three applications are incorporated herein by reference in their entirety. The application also claims the benefit of the priority of U.S. Provisional Application for patent assigned Ser. No. 61/318,291 filed on Mar. 27, 2010 which has been commonly assigned to the same assignee. This application also incorporates by reference U.S. Provisional Patent Application for Patent bearing Ser. No. 61/248,997, United States Patent Application bearing Publication No. 2006/0047281, PCT Application bearing Publication No. WO2009/072108, and U.S. Provisional Patent Application bearing Ser. No. 61/307,517.

### BACKGROUND

The method and apparatus generally relate to treating diseased nails, and more particularly to treating diseased nails using electro-magnetic radiation and/or ultrasound energy to substantially deactivate the source of the disease.

Thick, discolored, disfigured, and/or split nails can be common symptoms of disease of a fingernail or toenail. This disease can be caused by a variety of factors or elements, such as but not limited to, bacteria, mold, a fungus, viruses, parasites, or other organisms or microorganisms, and if left untreated, the disease can result in partial or complete destruction of a patient's nail plate. These diseases are chronic and difficult to treat and may sometimes even require surgical intervention such as debridement—the medical removal of a patient's diseased tissue or removal of the nail plate.

In general, the most common type of nail disease is onychomycosis or simply mycosis, which can be caused by a fungus, such as, a dermatophyte that can invade the nail plate and nail bed forming a patient's nail. Creams, ointments, oral medications, and radiation can be used to treat onychomycosis or other nail diseases. These treatments, however, may not eliminate the source of the disease, do not work for many patients, and can cause numerous side effects in patients. Thus, there is a need in the art for a method, system and/or device that more effectively treats onychomycosis.

### BRIEF SUMMARY

Various embodiments feature a method and apparatus to treat a diseased nail using electromagnetic radiation and/or other forms of energy. The treatment available by use and/or application of the various embodiments disclosed herein can eliminate, substantially eliminate or effectively reduce the source of disease in the nail. For instance, one of the results of the disclosed treatment is that an organism causing the disease can be deactivated. In one embodiment, the organism is thermally deactivated by delivering radiant electromagnetic radiation, radio frequency (RF) energy or ultrasound energy to a target area, which can be adjacent to the organism or can include the organism. Tissue surrounding the organism itself can absorb one or more of these energies generating thermal energy and transfer thermal energy to the organism to deactivate the organism, and/or the organism can absorb the energy directly. Deactivation of the organism can render it dormant or unable to grow, reproduce and/or replicate, and, in some embodiments, can destroy the organism.

In one aspect, an exemplary method of treating a diseased nail, wherein the nail includes a nail bed and a nail plate, includes delivering RF energy to a target area to thermally deactivate an unwanted organism without causing substantial, unwanted injury to the nail bed and/or the nail plate. The RF energy induced electric current flows through the nail bed tissue heating a segment of the tissue to a temperature level sufficient to substantially deactivate the organism without causing unwanted injury to either the nail bed tissue and/or nail plate. RF energy can be delivered to the target area by employing an applicator or hand piece including one or more energy applying elements, such as, for example, RF electrodes. The applicator can be automatically adjustable so that when a digit is properly inserted into the applicator, the electrodes come to rest on opposite sides of the digit nail plate or, on opposite sides of the nail bed tissue normally covered by the nail plate. RF energy may be delivered simultaneously to the entire target area or by sequentially or randomly sweeping or scanning segments of the target area. In addition, the RF energy may be delivered in a continuous delivery mode or in a pulse delivery mode. The electrical current induced by the RF voltage between the electrodes flows through the nail bed tissue, heating a segment of the nail bed tissue to a temperature level sufficient to substantially thermally affect mycoses pathogens therein without causing unwanted injury to either the nail bed tissue and/or nail plate. Heating of the nail bed tissue in such a manner has an adverse affect on the mycoses pathogens such as, but not limited to, deactivating, retarding, and or killing off the mycoses pathogens.

In accordance with another exemplary embodiment of the current method and apparatus, the applicator may also include a source, or be connected to a source, of radiant electromagnetic energy or light generating a beam of radiant energy and irradiating the nail bed tissue through the nail plate. The radiation may be applied concurrently or alternately with the application of RF energy by the RF electrodes, heating the nail bed tissue. Alternatively, the nail bed tissue may be irradiated by radiant energy only.

In accordance with yet another embodiment of the current method and apparatus, the RF electrodes may include one or more voltage-applying-elements-carriers having on one surface thereof a plurality of voltage-applying-elements in a spaced apart pattern and apply RF voltage to the nail bed tissue in a linear sweeping wave effect. Alternatively, the spaced-apart elements may be light emitting elements such as LEDs, VCSELs, laser diodes, laser diode bars, and others.

Another exemplary embodiment of the present method of treating a diseased nail, the diseased nail having a nail bed and a nail plate, includes delivering mechanical energy, such as ultrasound wave energy, to a target area. The energy absorbed is converted to thermal energy that is trapped by the nail bed tissue or nail plate of the diseased nail. An unwanted organism in at least one of the nail bed and the nail plate can be thermally deactivated without causing substantial unwanted injury to the nail bed and/or the nail plate of the diseased nail. The temperature in the region where the unwanted organism resides can be raised sufficiently to deactivate the organism, but not high enough to result in unwanted injury to the surrounding tissue.

In order to deliver ultrasound energy to the nail bed, the RF electrodes may be replaced or supplemented by one or more ultrasound transducers positioned on opposite sides or on one side only of the nail plate and/or the nail bed tissue and be operative to emit an ultrasound beam (and/or RF beam) into a segment of the nail bed tissue at a wavelength and power sufficient to substantially thermally affect the organism without causing injury to either the nail bed tissue and/or the nail plate.

In another exemplary embodiment, the ultrasound is delivered by one or more ultrasound transducers positioned on opposite sides of the nail plate and oriented to couple ultrasound to the treated target at a Brewster angle such that most of the tissue heating concentrates in the nail bed tissue. The heating should be sufficient to substantially thermally affect the unwanted organisms which may be mycoses pathogens without causing injury to either the nail bed tissue and/or the nail plate.

The energy delivery elements of the apparatus for treating a diseased nail, having a nail plate and a nail bed, such as electrodes, light sources or lasers, and/or ultrasound transducers communicate with an energy source, and deliver the energy provided by the energy source to a target area. An unwanted organism in the nail bed and/or the nail plate is thermally deactivated or destroyed without causing substantial unwanted injury to the nail bed and/or the nail plate of the diseased nail.

In accordance with another embodiment of the current method and apparatus, the applicator may also include one or more digit contacts and/or digit positioning sensors to ensure correct placement of a digit in the applicator and optimal placement of the energy providing electrodes, transducers, and source of radiant energy prior to initiation of treatment. In some embodiments, the sensors may be selected from a group consisting of micro switches and light detectors.

In accordance with the current method and apparatus, any one of the embodiments mentioned hereinabove may also include one or more sensors operative to sense the treated digit temperature and one or more temperature change indicating indicators.

The temperature change indicating indicators, in some embodiments, may be selected from a group consisting of impedance and ultrasound wave propagation speed sensors.

The treatment can be performed by a caregiver a single time on a diseased nail, or, in some embodiments a plurality of treatments may be required. The treatments can also be performed by a “layman” consumer in a residential environment. The treatments can be followed by the application of topical cream or an ointment to the diseased nail, the cuticle, and/or surrounding tissue or by administering a medication (e.g., oral or intravenous) to prevent reoccurrence of the unwanted organism or to eliminate the unwanted organism.

Other aspects and advantages of the method and apparatus will become apparent from the following drawings, detailed description, and claims, all of which illustrate the principles of the method and apparatus, by way of example only.

### BRIEF DESCRIPTION OF THE SEVERAL VIEWS OF THE DRAWING

The present method and apparatus will be understood and appreciated from the following detailed description, taken in conjunction with the drawings. In the drawings, like reference characters generally refer to the same parts throughout the different views. The drawings are not necessarily to scale, emphasis instead generally being placed upon illustrating the principles of the invention.

FIG. 1 depicts a schematic view of an exemplary apparatus for treating a diseased nail according to the present method;

FIG. 2 shows a schematic view of an exemplary hand piece for holding an appendage having a diseased nail during treatment according to the present method and apparatus;

FIG. 3 shows a sectional view of another exemplary hand piece for holding an appendage having a diseased nail during treatment according to the present method and apparatus;

FIG. 4 is a simplified frontal view and cross-sectional view illustration of another exemplary embodiment of the current energy to a diseased nail hand piece according to the present method and apparatus;

FIG. 5 is simplified cross-sectional view of another exemplary embodiment of the current energy to a diseased nail applicator (or hand piece) according to the present method and apparatus;

FIG. 6 is simplified cross-sectional view of a modified exemplary embodiment of the current energy to a diseased nail applicator of FIG. 5 according to the present method and apparatus;

FIG. 7 is a simplified plan view illustration of an RF electrode suitable to generate a linear sweeping wave effect in accordance with yet another exemplary embodiment of the current method and apparatus;

FIGS. 8A, 8B, 8C, 8D, and 8E are simplified cross-sectional view illustrations of a linear sweeping wave effect in accordance with yet another exemplary embodiment of the current method and apparatus;

FIG. 9 is a simplified cross-sectional view illustration of still another exemplary embodiment of the current energy to a diseased nail applicator according to the present method and apparatus;

FIG. 10 is a simplified cross-sectional view illustration of a further exemplary embodiment of the current energy to a diseased nail applicator according to the present method and apparatus.

### DETAILED DESCRIPTION OF EXEMPLARY EMBODIMENTS

For the purposes of this disclosure, the term “Digit” or “Appendage” as used below means a finger, a digit or both but, those skilled in the art will appreciate that the digits may include fingers and/or toes although the treatment is not necessarily limited thereto.

For the purposes of this disclosure, the term “Controller” as used below means a hardware and software unit controlling the treatment process, regulating treatment energy supply and timing, and other apparatus functions.

For the purpose of this disclosure, the term “light” means laser light or radiation, Intense Pulsed Light (IPL), Continuous light illumination, and Low Level Light Treatment Therapy (LLLT).

For the purpose of this disclosure, the term “types of treatment energy” means treatment energy intended for producing a desired result. It may be a light or radiant energy, RF energy, ultrasound energy, and a combination of the above types of energies.

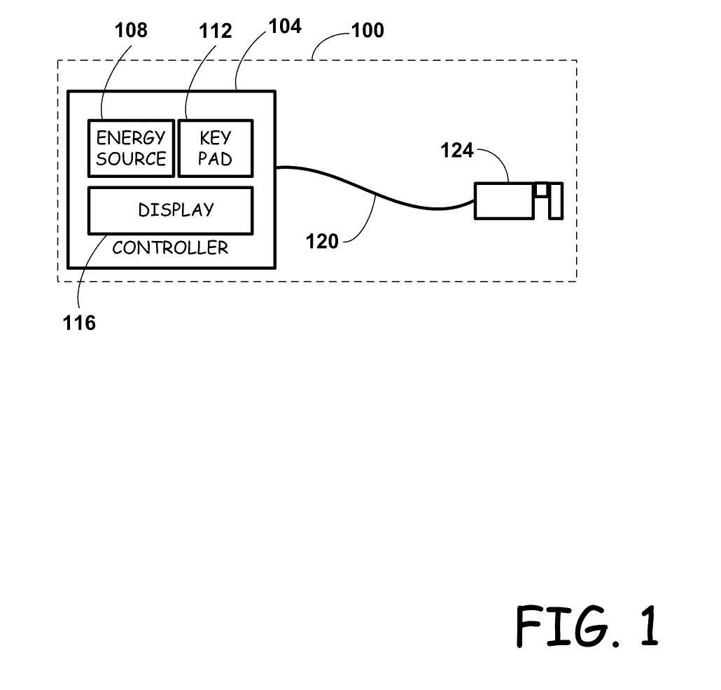

FIG. 1 shows an exemplary embodiment of a system 100 for treating a nail having a disease. The system 100 includes a controller 104 that incorporates an energy source 108, a keypad 112 for entering treatment instructions, a display 116 displaying treatment parameters and status, and a delivery cable 120 communicating with hand piece or applicator 124. The treatment energy provided by the energy source 108 is directed by the hand piece to a target region of a diseased nail or a nail bed. The target region can be a target area, target segment, or a target volume of tissue.

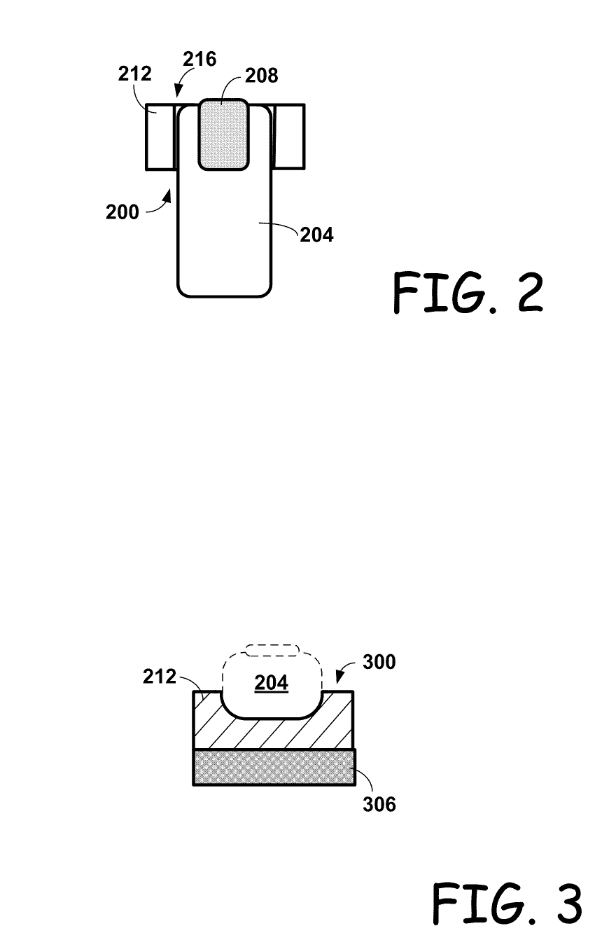

FIG. 2 shows an exemplary hand piece or applicator 200 for positioning a patient's appendage or digit 204 (e.g., a finger or a toe) having a diseased nail 208. The applicator 200 includes a base 212 defining an opening 216 for retaining or cradling the appendage 204 during a treatment.

FIG. 3 shows an exemplary embodiment of a hand piece 300 including a cooling pad 306. Cooling generally, can facilitate a treatment and reduce, to some extent, a patient's sensitivity to pain. The cooling pad 306 can be placed below the appendage 204, or can be affixed to the base 212 to cool the appendage 204 shown in broken lines. The cooling pad 306 can be filled with ice, a frozen gel pack, or a cooling fluid made to circulate through it. In addition, the cooling pad 306 can be structured using heat sinking technology based on highly heat conductive material that likewise can operate to cool the appendage 204. In operation, controller 104 operates energy source 108 (FIG. 1) such that energy desired for the treatment of the nail is delivered through delivery system 120 to the appendage 204 having the diseased nail 208. The energy can be a variety of types or, combinations thereof and as such, may include radiant energy or another type of treatment energy. Controller 104 may also activate delivery of cooling fluid, which may be delivered through tubing being part of delivery cable 120. Cooling of the nail plate before, during or after delivery of electromagnetic radiation minimizes thermal injury to tissue surrounding the diseased nail 208. Cooling can include contact conduction cooling, evaporative spray cooling, convective air flow cooling, or a combination of the aforementioned.

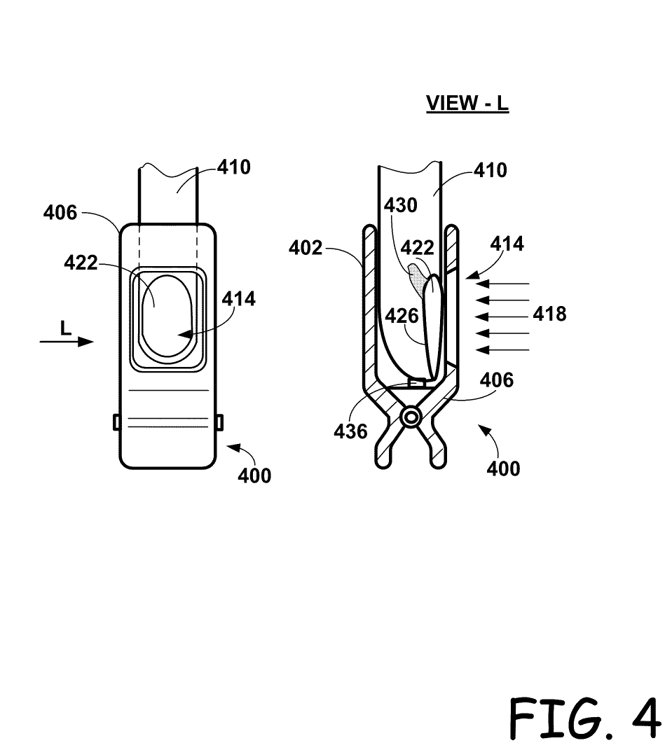

FIG. 4 is a simplified frontal view and cross-sectional view illustration of another exemplary embodiment of the current hand piece. Hand piece 400 has a base 402 and a cover 406, operative to comfortably accommodate a subject's digit 410, i.e., finger or toe. In one embodiment, hand piece 400 may have an aperture or window 414 made in cover 406 to allow the tissue being treated to be exposed to radiant electromagnetic energy such as light, shown schematically by arrows 418 through nail plate 422, to nail bed tissue 426 and nail root permeated by one or more organisms schematically shown by numeral 430, when a subject's digit is fully inserted in hand piece 400, as will be explained in detail below.

The radiant energy applied by the light source and schematically shown by arrows 418 may characteristically have a wavelength in excess of about 400 nm, commonly being in the range between about 400 nm and about 2600 nm with an effective fluency in the range of about 2 J/cm² to about 50 J/cm² and higher, and a spot size in the range between about 2 mm² and about 200 mm². The radiant or light energy source can be an incoherent light source (e.g., IPL) or a coherent light source (e.g., a laser). In some embodiments, two or more different radiant energy sources can be used together to effect a treatment. For example, an incoherent source can be used to provide a first beam of radiation while a coherent source provides a second beam of radiation. The first and second beams of radiation can share a common wavelength or can have different wavelengths. In an embodiment using an incoherent light source or a coherent light source, the beam of radiation can be a pulsed beam, a scanned beam, or a gated CW beam.

Exemplary coherent light sources such as lasers include, but are not limited to, pulsed dye lasers, Nd:YAG lasers, frequency doubled Nd:YAG lasers, Nd:glass lasers, copper vapor lasers, alexandrite lasers, frequency doubled alexandrite lasers, titanium sapphire lasers, ruby lasers, fiber lasers, and diode lasers. Exemplary pulsed dye lasers include V-Beam brand lasers and C-Beam brand lasers, both of which are available from Syneron Medical Ltd., (Formerly Candela Corporation (Wayland, Mass.). Exemplary incoherent light sources include, but are not limited to, intense pulsed light sources, arc lamps, flash-lamps (e.g., an argon or xenon lamp), filament lamps, and light emitting diodes. In various embodiments, the beam of radiation can have a fluence between about 1 J/cm² and about 50 J/cm², although higher and lower fluences can be used depending on the application. In some embodiments, the fluence can be between about 2 J/cm² and about 20 J/cm².

In various embodiments, the beam of radiation can have a spot size between about 1 mm² and about 50 mm², although larger and smaller spot sizes can be used depending on the application. In some embodiments, the spot size can be between about 2 mm² and about 200 mm².

In various embodiments, the beam of radiant energy can have a pulse width between about 10 microseconds and about 30 seconds or even be continuous (CW), such as in LLLT, although larger and smaller pulse widths can be used depending on the application. In some embodiments, the pulse width can be between about 100 microseconds and about 1 second. In one detailed embodiment, the pulse width can be about 100 microseconds, about 500 microseconds, about 1 milliseconds, about 5 milliseconds, about 10 milliseconds, about 50 milliseconds, about 100 milliseconds, about 500 milliseconds, or about 1 second.

In various embodiments, the beam of radiation can be delivered at a rate of between about 0.1 pulse per second and about 10 pulses per second, or CW, although faster and slower pulse rates can be used depending on the application. In one detailed embodiment, the pulse rate is about 1 pulse per second.

In various embodiments, the pulsed beam of radiation is scanned over the surface of the disease nail during a treatment. That is, the beam of radiation can be moved after and during delivery of one or more pulses. In one embodiment, the beam of radiant energy is moved after a single pulse. The pulse rate can be one pulse per second, although other suitable pulse rates can be used. The beam of radiant energy can be moved or scanned until the entire surface of the diseased nail has been substantially completely irradiated.

To increase the safety of use, the current method and hand piece incorporates safety features such as thermo-sensors, which are operative to sense nail bed tissue temperature, as well as digit contact and/or digit positioning or alignment sensors to ensure correct placement, relative to energy applying element which in this case is a beam of light or radiant energy 418, of at least a portion of digit 410 inserted into applicator 400. One example of such sensors may be a contact sensor or a micro switch 436. Contact sensor 436 may become operative when a digit 410 is fully inserted into applicator 400, pressing against and activating contact sensor 436 to indicate proper digit positioning. Activation of contact sensor 436 activates a source of light to generate beam 418 and irradiate nail bed tissue 426 through aperture 414. Both base 402 and cover 406 may have a channel for retaining or cradling the digit 410 during a treatment. (As will be explained below sensor 436 or a similar one may be used in a similar manner for ultrasound treatment.)

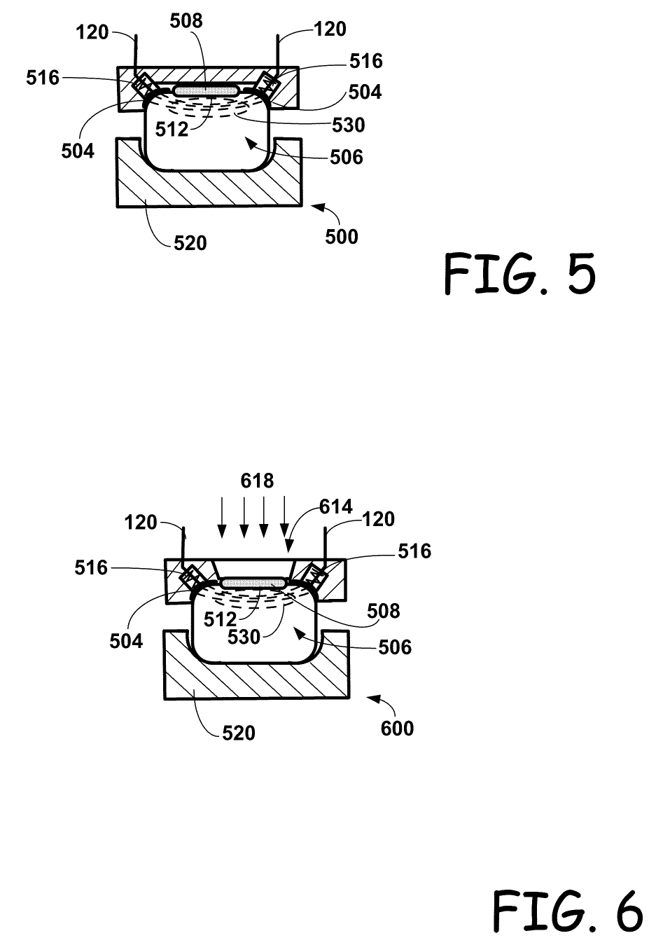

FIG. 5 is simplified cross-sectional view of another exemplary embodiment of the current energy to a diseased nail applicator according to the present method and apparatus. Hand piece or applicator 500 includes one or more RF electrodes 504 connected by energy delivery cable 120 to controller 104 (FIG. 1) and source of energy 108. The distance between the electrodes may be automatically adjustable so that when a digit 506 is inserted into applicator 500 electrodes 504 come to rest on opposite sides of the digit 506 nail plate 508 or, in the lack thereof, on opposite sides of nail bed tissue 512, normally covered by a nail plate 508. For this purpose, electrodes 504 may be, for example, spring-biased by springs 516 or adjustable by other suitable means. Electrodes 504 may be adjustable to accommodate digits of various sizes as well as to enable proper coupling of the electrodes 504 to the skin. Numeral 520 marks the base of applicator 500 and numeral 530 marks the segment or volume of tissue heated by the RF induced current and affecting nail bed tissue 512.

FIG. 6 is a simplified cross-sectional view of a modified exemplary embodiment of the current energy to a diseased nail applicator of FIG. 5 according to the present method and apparatus. Applicator 600 is generally similar to applicator 500 but differs from applicator 500 in that it has an aperture, opening or window 614 enabling application of radiant energy 618 to a diseased nail. The light energy and the RF energy may be applied concurrently, sequentially or in partially overlapping in time order. The second type of energy is activated to accelerate receiving a desired result. Each of the energies may be applied in pulse or continuous operation mode. Alternatively, nail bed tissue 512 may be irradiated without the application of RF energy. It is known (see “Physical Properties of Tissue” by Francis A. Duck, Academic Press, 1990, page 200) that tissue conductivity is temperature dependent, thus when light is applied to the diseased nail first it heats the diseased nail and in particular the immediate tissue beneath the dark colored nail, forming a preferential path for RF induced current. The radiation applied by light source and schematically shown by arrows 618 may characteristically have a wavelength in excess of about 400 nm, commonly being in the range between about 400 nm and about 2000 nm with an effective fluency in the range of about 2 J/cm² to about 20 J/cm² and higher and a spot size in the range between about 2 mm² and about 200 mm².

In one embodiment, the hand piece 200 includes a nail contacting portion that can be brought into contact with the region of the diseased nail receiving the beam of radiation. The nail contacting portion can cool the nail plate. The nail contacting portion can include a sapphire or glass window and a fluid passage containing a cooling fluid. The cooling fluid can be a fluorocarbon type cooling fluid, which can be transparent to the radiation used. The cooling fluid can circulate through the fluid passage and past the window contacting the nail plate.

Applicators 500 and/or 600 may incorporate safety features such as thermo-sensors as well as digit contact and/or digit positioning sensors to ensure correct placement of a digit in applicator 500 or 600 and optimal placement of electrodes 504 and source of light 618 prior to initiation of treatment. For example, since most of the heat is generated in nail bed tissue 512, which is normally covered by nail plate 508, the measurement of temperature changes therein by conventional thermo-sensors such as thermistors or thermocouples may be difficult. Hence, temperature changes in nail bed tissue 512 may require indirect measurement thereof by employing indicators such as nail bed tissue impedance or changes in ultrasound wave propagation speed, affected by tissue temperature changes, as described in assignee's U.S. Provisional Patent Application for Patent bearing Ser. No. 61/248,997, the disclosure of which is hereby incorporated by reference. Alternatively an IR based remote thermal sensor can be used to monitor the temperature of the nail bed. In this case, the IR wavelength range used to make the temperature measurement should be in the region where the nail plate has low absorption.

A contact sensor, similar to sensor or micro switch 436 (FIG. 4) may be incorporated into applicators 500 and 600. The sensor may become operative when a digit 506 is fully inserted into applicator 500 or 600, pressing against and activating contact sensor to indicate proper digit positioning. Activation of the contact sensor activates or enables source of light energy to generate beam 618 and irradiate nail bed tissue 512 through aperture 614. Additionally or alternatively, the contact sensor may activate or enable RF energy application to nail bed tissue 512 by RF electrodes. Withdrawing digit 506 from applicator 500 or 600 releases pressure from the contact sensor and may bring about immediate cessation of the application of all forms of energy.

Contact sensor similar to sensor 436 (not shown) may be a spring-biased micro-switch, a capacitive sensor or any other sensor operative to detect contact of the tip of finger 506 with applicator 500 or 600.

Additionally or alternatively, other positioning sensors may be employed, located, for example, in propinquity with electrodes 108 and engaging opposite sides of nail bed tissue 512 and/or digit 510, to ensure correct placement of electrodes 504 with the skin/digit, and other digit contact and/or digit positioning sensors.

As shown in FIGS. 5 and 6, RF electrodes 504 rest on opposite sides of nail plate 508 and, nail bed tissue 512 to be treated is normally covered by nail plate 508. Because nail plate 508 is made of Keratin, a highly non-conductive material, the electrical current induced by voltage between RF electrodes 504 flows through the shortest path of travel, which, in this case, is through a segment 530 of nail bed tissue 512, which includes one or more layers of skin, heating segment 530 to a temperature level sufficient to substantially thermally affect the undesired organisms, which may be mycoses pathogens, without causing substantial unwanted injury to either nail bed tissue 512 and/or nail plate 508. The applied RF energy may be supplied in continuous and/or pulse mode and may be in the range between 300 KHz and 40 MHz.

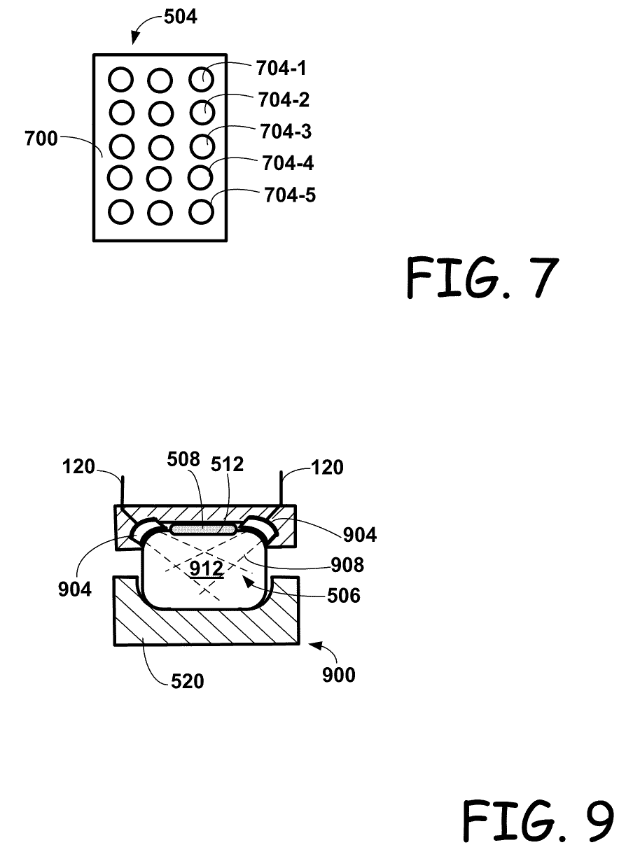

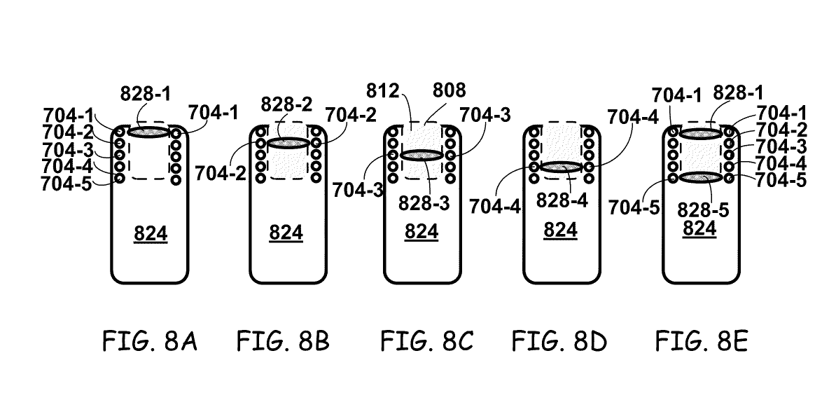

Reference is now made to FIGS. 7, and 8A, 8B, 8C, 8D, and 8E, which are simplified cross-sectional views and plan view illustrations of a linear sweeping wave effect and electrode architecture suitable to produce such effect in accordance with yet another exemplary embodiment of the current method and apparatus.

The level of heat generated by the forms of energy described hereinabove sufficient to affect the unwanted organisms may be above 50 degrees Celsius requiring the cooling of the tissue to be treated between periods of energy application. This may be achieved as explained above by a cooling arrangement shown in FIG. 2 or alternatively, by actively cooling electrodes 504 and tissue to be treated employing cooling fluid circulating through tubes attached to electrodes 504 as described in assignee's United States Patent Application bearing Publication No. 2006/0047281. Alternatively, cooling may be achieved by allowing the tissue sufficient time, longer than the thermal relaxation time of skin, to cool.

As shown in FIG. 7, electrodes 504 may be of the type described in assignee's PCT Application bearing Publication No. WO2009/072108, employing a linear sweeping heating wave effect as described in assignee's U.S. Provisional Patent Application bearing Ser. No. 61/307,517 the disclosures of which are hereby incorporated by reference.

In FIG. 7, RF electrode 504 may include one or more voltage-applying-elements-carriers 700 having on one surface thereof a plurality of voltage-applying-elements 704 in a spaced apart pattern, for example, arranged along rows (704-1), (704-2), (704-3), (704-4) and (704-5). Alternatively, elements 704 may be light emitting elements such as LEDs, VCSELs, laser diodes, laser diode bars, and others.

FIGS. 8A 8B, 8C, 8D, and 8E illustrate a linear sweeping wave effect in accordance with yet another exemplary embodiment of the current method and apparatus. Electrodes 704 may be placed on opposing sides of a nail bed 812 and/or nail plate 808 and be connected to a source of power 108 of controller 104. Each of electrodes 704 may include one or more voltage-applying-elements-carriers 700 having on one surface thereof a plurality of voltage-applying-elements 704.

In FIG. 8A, for example, two voltage-applying-elements-carriers (for the simplicity of explanation only voltage-applying-elements 704 are shown) are positioned on either side of nail bed or nail plate 808 of digit 824. In FIGS. 8A-8E nail plate 808 has been shown in broken lines for illustrative purpose only.

The controller may be operative to activate RF voltage applying elements 704 of rows (704-1), (704-2), (704-3), (704-4) and (704-5) in an order set by a predetermined protocol so as to generate a linear sweeping heating wave effect. For example, in FIG. 8A, controller 104 (FIG. 1) has activated voltage applying elements 704 of carriers (not shown) in row (704-1) only, as indicated by the blackening 828-2. RF current flows from elements 704 in row (704-1) of one carrier, through tissue to be treated, to elements 704 in row (704-1) of the other carrier, heating the blackened zone 828-1 of a respective segment of nail bed tissue 712.

As illustrated in FIG. 8B, the controller has activated voltage applying elements 704 in row (704-2) only, as indicated by the blackening 828-2. RF current flows from elements 704 in row 704-2 through tissue to be treated, to elements 704 in row 704-2, heating the respective blackened zone 828-2 of nail bed tissue 812.

In FIG. 8C, the controller has activated voltage applying elements 704 of row (704-3) only, as indicated by the blackening 828-3. RF current flows from elements 704 in row 704-3 through tissue to be treated, to elements 704 in row 704-3 heating a blackened zone 828-3 of nail bed tissue 812 located between the respective elements.

As shown in FIG. 8D, the controller has activated voltage applying elements 704 in row 704-4 only, as indicated by the blackening 828-4. RF current flows from elements 704-4 on one side of the digit through tissue to be treated to elements 704-4 located on the other side of the digit heating a blackened zone 828-4 of nail bed tissue 812 located between the electrodes.

In FIG. 8E, the controller has activated voltage applying elements 704 of in row 704-5, as indicated by the blackening 828-5 thereof. RF current flows from elements 704-5 through tissue to be treated, to elements 704-5 heating a respective zone 828-5 of nail bed tissue 812.

The serial application of RF energy results in an effect of linear progression of heated zone 828 through nail bed tissue 812, creating a linear sweeping tissue heating wave effect without physical or mechanical movement of, for example, applicator 100 or electrodes 108. This allows forming highly localized zones of rapid heating and cooling.

Additionally, maintaining an appropriate time interval between consecutive sweeping tissue heating wave effects applied to the same heated zone/segment or volume 828 longer than the thermal relaxation time of human skin allows natural cooling thereof, preventing overheating of the skin for unsafe extended periods of time and causing discomfort to the subject. Pain sensitivity threshold is different for different subjects. In order to provide optimal treatment; the interval between the successive sweeping tissue heating waves may be made adjustable. For instance, in some embodiments, a user may adjust a setting, such as an interval setting. In other embodiments, the interval may be dynamically adjusted based on a form of feedback. For instance, a temperature sensor may detect the relative heating of the tissue and either retard or increase the interval timing accordingly. More particularly, if the temperature is rising too rapidly or if it crosses a threshold, the interval may be decreased. Likewise, if the temperature is not changing rapidly enough or if it is not at a desired level, the interval can be increased. Similarly, either in conjunction with or alternatively to, the energy level applied may also be either manually adjustable or dynamically adjustable.

The direction of progression of the sweeping tissue heating wave effect described hereinabove is given with respect to the drawing plane. It is appreciated that the apparatus may not be limited to any particular plane and may be operative in any direction and orientation.

It will be appreciated by persons skilled in the art that the sweeping tissue heating wave effect may also be generated by employment of a source of light or radiant energy where a scanning laser beam or a matrix of laser diodes, LEDs or VCSELs may be used for this purpose.

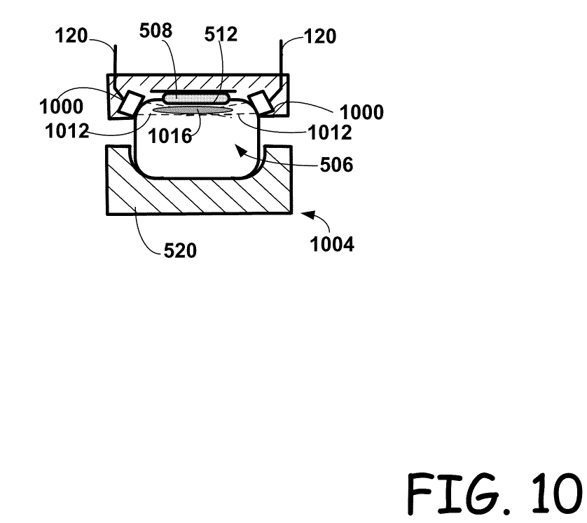

Reference is now made to FIG. 9, which is a simplified cross-section view illustration of still another exemplary embodiment of the current method and apparatus. In this exemplary embodiment of hand piece or applicator 900, RF electrodes 504 (FIG. 5) may be replaced by one or more ultrasound transducers 904 positioned on opposite sides of nail plate 508 and/or nail bed tissue 512. Either one or both of ultrasound transducers 904 may emit an ultrasound beam 908 into a segment or volume 912 of nail bed tissue 512 at a wavelength sufficient to substantially thermally affect the unwanted organisms which may be mycoses pathogens or other organism, without causing substantial unwanted injury to either nail bed tissue 512 and/or nail plate 508.

Alternatively, transducers 904 may be placed in propinquity to RF electrodes 504 (FIG. 5) and apply ultrasound beams concurrently and/or alternately with the application of RF energy to nail bed tissue segment 512. Optionally, transducers 904 may apply ultrasound beams concurrently and/or alternately with the application of a beam of radiant energy generated by the source of light radiation irradiating nail bed tissue segment 512 through nail plate 508. An optional opening (not shown) similar to openings 212, 414, or 812 enabling light radiation to nail 508 application could be made in applicator 900.

Because most of the heat should be generated in nail bed tissue 512, or very close to it (i.e. segment 912), which is normally covered by nail plate 508, the measurement of temperature changes therein by conventional thermo-sensors such as thermistors or thermocouples may be difficult. Hence, temperature changes in nail bed tissue 512 may require indirect measurement thereof by employing indicators such as nail bed tissue impedance or changes in ultrasound wave propagation speed, affected by tissue temperature changes, as described in assignee's U.S. Provisional Patent Application No. 61/248,997, the disclosure of which is hereby incorporated by reference.

In accordance with the current method and apparatus, any one of the embodiments mentioned above may also include one or more heat monitoring mechanisms operative to receive and analyze temperature and one or more temperature change indicators selected from a group consisting of impedance and ultrasound wave propagation speed.

Ultrasonic energy directed to a diseased nail or nail bed can be substantially reflected at the tissue layers interface between the nail plate and the nail bed. As a result, the nail bed will not be as strongly heated as the nail plate-nail bed tissue layers interface by the ultrasound energy. Moreover, in general, the nail plate has a higher attenuation or absorption than the underlying nail bed. Thus, a high intensity, focused ultrasound device applied to the nail plate by a transducer placed over the surface of the diseased nail effectively heats up the nail plate without causing excessive thermal damage to the underlying nail bed.

In FIG. 10, which is a cross sectional view illustration of another embodiment of the current method and apparatus, an ultrasound transducer 1000 of applicator 1004, is operative to emit ultrasound beams, commonly in pulse or continuous mode, at an adjustable angle (not shown) relative to the surface of nail bed tissue 512 to be treated. At least a portion of the beams emitted at this adjustable angle impinges upon the surface of nail bed tissue 512 at a predetermined angle of incidence also known as Brewster's angle of incidence. This causes total internal reflection of the ultrasound and enables propagation of the ultrasound in a desired nail bed tissue layer 512, as described in the U.S. Provisional Patent Application for Patent bearing Ser. No. 61/248,997, the disclosure of which is hereby incorporated by reference.

In light of the principle that ultrasound beams 1012 introduced into tissue at a Brewster's angle of incidence propagates generally along the tissue layers interface between two mediums having two different sound refraction indexes, part of the ultrasound beams propagate along the surface of nail bed tissue 512 and parallel thereto, heating segment or volume 1016. Ultrasound wave or beam emitted by one of the transducers 1000 may be eventually received by another optional ultrasound transducer 1000 that may operate as a transmitter or may be switched to operate as a receiver for treatment control purposes. The received signals may then be communicated to controller 104 (FIG. 1).

The controller is operative to obtain from the received ultrasound beam, and analyze, signaling information regarding changes in propagation speed of the beams, which are indicative of the temperature changes in nail bed tissue 512 through which the beams have propagated. The controller may change treatment parameters, or stop the treatment altogether, based on the temperature changes and a predetermined treatment protocol.

The ultrasound transducers may be of a conventional or phased array type. The transducers may be organized in a pattern/array similar to the pattern of RF electrodes shown in FIG. 8. Serial application of ultrasound energy to each of the array transducers will result in an effect of linear progression of heat, generated by the ultrasound, moving zone by zone through nail bed tissue. This process creates a linear sweeping tissue heating wave effect without physical or mechanical movement of, for example, the applicator or transducers. This allows forming, in immediate proximity to the nail plate tissue, highly localized zones of rapid heating and cooling.

The safety features of different embodiments described above are mutatis mutandis applicable to the current embodiment. Additionally or alternatively, other positioning sensors may be employed, located, for example, in propinquity with electrodes or transducers and engaging opposite sides of nail bed tissue and/or nail plate, to ensure correct placement of electrodes or transducers thereon, and other digit contact and/or digit positioning sensors.

One or more treatments can be followed by the application of a topical cream or an ointment to the diseased nail, the cuticle, and/or surrounding tissue or by administering a medication (e.g., oral or intravenous) to prevent reoccurrence of the unwanted organism or to eliminate the unwanted organism. In one embodiment, a diseased nail can be scraped to remove excess growth prior to applying energy to the diseased nail.

In some embodiments, the energy or fluence of the beam of radiation is predetermined so as to thermally destroy the particular source of the disease without causing substantial adverse side effects for the patient or substantial injury to surrounding tissue. In one embodiment, the fluence is selected after determining the source and/or size of the infection. Accordingly, the fluence can be tuned to preferentially heat the source of the disease without damaging or substantially injuring surrounding tissue.

In general, substantially all of the electromagnetic radiation within a broad wavelength range can be transmitted through a diseased nail in the early stages of infection. If an infection has progressed and the diseased nail is cloudy, yellow, or thick, a portion of the diseased nail can be scraped away, and/or an index matching solution can be applied to the diseased nail to improve its clarity. The index matching solution can be introduced to or infused in a porous region of the nail. For example, a porous nail can appear cloudy due to a difference in the index of refraction between air in the pores and the solid nail. To reduce the cloudiness and improve light transmission through the nail, the index matching solution can be applied to the nail to fill the voids, thereby decreasing the difference in the index of refraction. In general, index matching solutions are transparent fluids, such as, for example, water, glycol (e.g., ethylene glycol), glycerin, and mineral oil.

In some embodiments the diseased nail plate can be thinned by mechanically scrapping away a layer of the nail plate prior to treatment. Thinning the nail plate will facilitate the cooling of the nail plate. Thinning the nail plate will also facilitate the induction of RF current in the nail bed by RF electrode placed on the surface of the nail plate.

In some embodiments the digit with the diseased nail can be soaked in water for a period of time prior to treatment to increase the water content of the nail plate. Increasing the water content of the nail plate will increase the dielectric constant of the nail plate thereby facilitating capacitive coupling of RF energy through the nail plate into the nail bed. Increasing the water content in the nail plate will increase the thermal conductivity of the nail plate, which will facilitate cooling of the nail plate.

While the invention has been particularly shown and described with reference to specific illustrative embodiments, it should be understood that various changes in form and detail may be made without departing from the spirit and scope of the invention as defined by the appended claims.

What is claimed is:

1. An apparatus for treating a diseased nail area of a digit, said apparatus comprising:
a source of energy that can be applied to the diseased nail area for treatment of the diseased nail area;
an applicator, including at least one of a group of energy applying elements consisting of RF electrodes, ultrasound transducers, and openings accepting light energy; and
a digit alignment sensor operative to accept a digit and provide an indication that at least a portion of the digit is in a defined configuration relative to the energy applying elements.

2. A method of treating a diseased area of a nail on a digit, said method comprising:
providing an applicator configured to apply at least one type of treatment energy to a particular location on the digit, said applicator comprising:
treatment energy providing terminals; and
an alignment sensor adapted to sense and guide a portion of a digit in a defined configuration relative to said energy providing terminals; and
applying at least one type of treatment energy to at least a portion of the digit corresponding to the portion that is placed in a known configuration relative to the energy providing terminals.

3. A method of treating a diseased nail of a digit, said method comprising:
providing an applicator operative to apply to the digit at a predetermined location at least one type of treatment energy, said applicator including;
treatment energy providing terminals; and
an alignment sensor adapted to sense and guide a portion of a digit in a defined configuration relative to said energy providing terminals;
applying at least one type of treatment energy to at least a portion of the digit; and
creating a linear sweeping tissue heating wave effect without physical or mechanical movement of the applicator or energy providing elements.

4. The method according to claim 3, further comprising employing said linear sweeping tissue heating wave to form localized zones of rapid heating.

5. An apparatus for use in the treatment of skin mycoses, said apparatus comprising:
one or more energy sources providing electromagnetic and mechanical types of treatment energy;
an applicator for applying one or more of said types of treatment energy to at least one of a nail plate and nail bed tissue normally covered by a nail plate to heat said nail plate and nail bed tissue to a temperature sufficient to adversely affect skin mycoses pathogens; and
at least one temperature monitoring mechanism operative to monitor temperature of said tissue normally covered by the nail plate.

6. The apparatus according to claim 5, wherein said source of electromagnetic energy is at least one type of energy selected from a group consisting of RF energy and radiant energy with wavelength between 400 nm and 2000 nm.

7. The apparatus according to claim 5, wherein the applicator is operative to apply radiant light energy with a wavelength between 400 nm and 2000 nm through said nail plate.

8. The apparatus according to claim 5, wherein the applicator is operative to apply light radiation with a wavelength between 400 nm and 2000 nm to tissue normally covered by the nail plate.

9. The apparatus according to claim 5, wherein the source of mechanical energy is an ultrasound generator.

10. The apparatus according to claim 5, wherein said applicator also comprises at least two RF electrodes operative to couple RF energy to the skin and being adjustable to facilitate coupling of said electrodes to the skin.

11. The apparatus according to claim 5, wherein said electromagnetic energy is RF energy coupled to the skin by an applicator comprising at least two spring-biased RF electrodes where the distance between the electrodes is automatically adjustable.

12. The apparatus according to claim 5, wherein said RF energy is applied by an applicator comprising at least one RF electrode and wherein the electrode is at least one of a group consisting of continuous electrodes and an array of electrodes.

13. The apparatus according to claim 5, wherein the energy sources generate sufficient energy to heat said tissue sufficiently to affect said mycoses pathogens without causing substantial undesired damage to the tissue to be treated and surrounding tissue.

14. The apparatus according to claim 5, wherein the mechanical energy is applied by an applicator comprising at least one ultrasound transducer coupled to the tissue at an adjustable angle.

15. The apparatus according to claim 5, wherein the mechanical energy is applied by an applicator comprising at least one ultrasound transducer coupled to the tissue at an angle of incidence to cause propagation of at least a portion of the ultrasound wave in a desired tissue layer.

16. The apparatus according to claim 5, wherein the mechanical energy is applied by an applicator comprising at least one ultrasound transducer operative to apply ultrasound energy to said skin tissue normally covered by a nail plate and generate heat in said tissue sufficient to affect said pathogens.

17. The apparatus according to claim 5, wherein the mechanical energy is applied by an applicator comprising at least one ultrasound transducer operative to emit ultrasound beams into said tissue.

18. The apparatus according to claim 5, wherein the mechanical energy is applied by an applicator comprising at least one ultrasound transducer operative to emit ultrasound beams into said tissue and at least one transducer operative to receive the emitted ultrasound beams and communicate to a controller information regarding said emitted and received beams, so that said controller may analyze changes in propagation speed of said beams through said tissue to determine changes in temperature thereof.

19. The apparatus according to claim 5, wherein said applicator also comprises at least one heat monitoring mechanism operative to measure temperature of tissue to be treated and wherein said heat monitoring mechanisms is selected from a group of heat monitoring mechanisms consisting of acoustic, temperature and impedance sensors.

20. The apparatus according to claim 5, wherein said source of electromagnetic energy is at least one form of energy selected from a group consisting of RF energy and optical energy with wavelength between 400 nm and 2000 nm and wherein the frequency of the RF energy is in the range from 300 KHz to 40 MHz.

21. The apparatus according to claim 5, wherein the electromagnetic energy is applied by an applicator comprising at least one RF electrode operative to at least couple RF energy into said tissue and a heat monitoring mechanism operative to communicate to a controller information regarding said RF energy and tissue temperature, so that said controller may analyze changes in temperature of said tissue and adjust coupled RF energy levels in accordance with changes in said tissue temperature.

22. The apparatus according to claim 6, wherein said applicator also comprises at least one RF electrode including at least one carrier having on one surface thereof a pattern of a plurality of spaced apart RF voltage applying elements.

23. The apparatus according to claim 6, wherein further comprising at least one RF electrode including at least one carrier having on one surface thereof a pattern of a plurality of spaced apart RF voltage applying elements and a controller operative to employ said RF electrode to apply a sweeping tissue heating wave effect across said skin tissue normally covered by a nail to affect said mycoses pathogens.

24. The apparatus according to claim 6, wherein the time interval between consecutive periods of energy application to the same tissue segment is settable.

25. A method for use in the treating of skin mycoses pathogens, said method comprising:
applying one or more RF energy supplying electrodes proximate to soft tissue normally covered by a nail plate;
generating energy based at least in part on an RF induced current to heat at least a layer of skin tissue normally covered by a nail plate to a temperature sufficient to adversely affect said skin mycoses pathogens.

26. The method according to claim 25, further comprising the step of irradiating said skin tissue normally covered by a nail and generating heat in said tissue sufficient to affect said mycoses pathogens.

27. The method according to claim 25, further comprising the step of irradiating through a nail plate the layer of tissue located beneath said nail plate and further heating it.

28. The method according to claim 25, wherein the step of generating an RF induced current further comprises generating heat in said tissue at a sufficient level to affect said mycoses pathogens without causing substantial undesired damage to the tissue to be treated.

29. The method according to claim 25, wherein the step of generating energy comprises providing said energy in a continuous form.

30. The method according to claim 25, wherein the step of generating energy comprises providing said energy in a pulse form.

31. The method according to claim 30, wherein said step of applying said energy in pulse form further comprises applying said energy with a time interval between consecutive pulses of energy based on an adjustable setting.

32. The method according to claim 30, further comprising the step of monitoring temperature changes of tissue to be treated and, wherein said step of applying said energy in pulse form further comprises applying said energy with a time interval between consecutive pulses of energy based at least in part on the monitored temperature changes.

33. The method according to claim 25, further comprising the step of monitoring temperature changes of tissue to be treated.

34. The method according to claim 33, further comprising the step of adjusting treatment parameters in accordance with monitored changes in temperature of tissue to be treated and a predetermined treatment protocol.

35. The method according to claim 25, further comprising the step of applying ultrasound energy to said skin tissue normally covered by a nail and generating heat in said tissue sufficient to affect said mycoses pathogens.

36. The method according to claim 25, further comprising the step of employing ultrasound to emit and receive ultrasound beams into and from said tissue and analyzing changes in propagation speed of said beams through said tissue to determine changes in temperature thereof.

37. The method according to claim 25, wherein the step of generating energy further comprising generating a tissue heating sweeping wave effect across said skin tissue normally covered by a nail plate to affect said mycoses pathogens.

38. The method according to claim 37, further comprising the step of generating subsequent tissue heating sweeping wave effects across said skin tissue and, wherein the time interval between consecutive sweeping tissue heating wave effects applied to the same tissue segment is adjustable.

39. The method according to claim 37, wherein the time interval between consecutive sweeping tissue heating wave effects applied to the same tissue segment is greater than the thermal relaxation time of human skin.

40. The method according to claim 25, further comprising the step of applying ultrasound energy to said tissue layer normally covered by a nail plate and generating heat in said tissue sufficient to affect said skin mycoses pathogens.

41. A method for use in the treating of skin mycoses pathogens, said method comprising:
applying light energy to a diseased nail infected with mycoses pathogens and heating the nail soft tissue normally covered by a nail plate to a temperature higher than the surrounding tissue to form a preferential RF current path;
generating an RF induced current flowing through said preferential current path to heat at least a layer of skin tissue normally covered by a nail plate to a temperature sufficient to affect said skin mycoses pathogens.

42. A method for use in the treating of skin mycoses pathogens, said method comprising applying ultrasound energy to the diseased nail and heating soft tissue proximate to the nail and normally covered by a nail plate to a temperature sufficient to affect said skin mycoses pathogens.
